(19) United States
(12) Patent Application Publication
(10) Pub. No.: US 2002/0150575 A1
(43) Pub. Date: Oct. 17, 2002

(54) METHOD TO SUPPRESS APPETITE AND REDUCE WEIGHT GAIN

(76) Inventors: MARK E. COOK, MADISON, WI (US); DARIA L. STRANSKY, OWATONNA, MN (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 09/479,811

(22) Filed: Jan. 7, 2000

(51) Int. Cl. A61K 39/395; A61K 39/42; A61K 39/40

(52) U.S. Cl. 424/139.1; 424/145.1

(57) ABSTRACT

A method of suppressing appetite and reducing weight gain in animals and human beings includes the step of administering to the animal a high dose of an antibody to a gut peptide.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

This invention relates to controlling appetite and weight gain in animals and human beings by using antibodies to gut peptides. Specifically, this invention relates to suppressing appetite and reducing weight gain in animals and humans by feeding animals or humans a substance that contains sufficiently high doses of anti-cholecystokinin (CCK) antibodies for a sufficient long period of time.

The immune system, based on several kinds of specialized blood white cells, is a highly specific defense system that recognizes, eliminates and remembers foreign macromolecules and cells. While functioning properly, it can distinguish between “self” and “non-self” (foreign) materials. For example, it views tumor cells as non-self and hence attacks them, protecting animals against cancer-causing tumor cells as it protects against other invading macromolecules.

An antigen is a foreign substance that when introduced into an animal with a functioning immune system, can elicit a specific immune response such as the one mentioned above. Once activated the immune response involves, among other things, production of antibodies in the circulation system specific to that antigen. There are five distinct classes of antibodies which are also called immunoglobulins. The most abundant is IgG. The other four are IgM, IgA, IgD, and IgE. These antibodies combine with the antigen and act to neutralize or counter the effects of the antigen introduced into the animal. They accomplish this result by binding to the antigen thereby neutralizing it and preventing it from binding to other specific cell receptors.

There are several ways in which an animal becomes immune responsive. Some antibodies are able to traverse the placenta from a mother's circulation to that of her fetus. As a result, the progeny of that mother receives natural immune protection by “inheriting” the mother's own antibodies before birth.

A second way to elicit an immune response is through introduction of an antigen into one animal, resulting in that animal developing specific antibodies to that antigen. These antibodies can then be isolated from the animal and introduced into a second animal resulting in the second animal having antibody that can bind the specific antigen.

CCK is an octapeptide that negatively affects food intake and thus inhibit growth in both mammals (Gibbs et al, 1973) and birds (Savory and Hodgkiss, 1984). CCK is released as a polypeptide hormone when food enters the small intestine. The presence of CCK in the gut mucosa alters gastrointestinal (GI) motility. The gizzard controls the rate in which food travels through the intestine and CCK, which is normally released after a meal is consumed, causes a decrease in gizzard contraction and an increase in intestinal contraction. This results in less time for the absorption of food and nutrients in the intestinal tract. The presence of CCK also alters the willingness to eat. CCK is responsible for what is known as the satiety effect which is a physiological effect that sharply decreases an animal's appetite.

When antibody to CCK has been administered to an animal, it has been observed that the satiety effect is inhibited and adverse effects of endogenous CCK on gastrointestinal motility are averted. CCK antibodies have been produced endogenously in pigs (Pekas and Trout, 1990; Pekas 1991) and rats (MacLaughlin et al, 1985). In both species, the adverse effects of CCK on food intake and weight gain were prevented by endogenous circulation of CCK antibodies.

CCK antibodies can similarly increase food intake and weight gain in domestic fowls. Such effect has been achieved by two methods. One method involves inoculating a female avian with a specific antigen which results in passively transferring of antibodies generated towards the antigen to the female's progeny. This passive transfer of anti-CCK antibodies has been shown to lead to improved conversion of food into body weight in the progeny. The other method involves directly feeding poultry with a substance containing anti-CCK antibodies. Antibodies to CCK was produced in laying hens, passed to the yolk, harvested from the yolk or fed as dried yolk, and used as a feed additive for improving feed efficiency in poultry.

### BRIEF SUMMARY OF THE INVENTION

Low doses of anti-CCK antibodies, when administered to animals, reduce satiety and increase body weight gain. The present invention relates to feeding higher doses of anti-CCK antibodies to animals to achieve opposite effects, namely suppressing appetite and reducing weight gain.

It is the object of the present invention to feed animals with a substance containing a sufficiently high level of anti-CCK antibodies for a sufficient time to lead to the effect of suppressing appetite and thereby reduce weight gain.

The effect of suppressing appetite and reducing weight gain of relatively high dose of CCK antibody can be reversed by switching animals to a diet containing low dose of CCK antibody, which has been shown to increase food intake and weight gain. Thus, the current invention allows one to control (increase or decrease) food intake and weight gain as desired at various stages in an animal's life or development by choosing to feed the animal with a diet containing either low or high dose of CCK antibody.

### BRIEF DESCRIPTION OF THE SEVERAL VIEWS OF THE DRAWINGS

FIG. 1 shows the effect of anti-CCK antibody dosage on growth rate in chicks.

FIG. 2 shows the effect of anti-CCK antibody dosage on feed consumption by chicks.

### DETAILED DESCRIPTION OF THE INVENTION

This invention involves administering to an animal a sufficiently high dose of an antibody to cholecystokinin (CCK) for a sufficiently long time to suppress appetite and thereby reduce weight gain in the animal. The anti-CCK antibody can be mixed with a suitable ingestible carrier such as feed meal. The amount of CCK that brings about the claimed effect is at least about three times as high as the amount known to reduce satiety and increase weight gain in an animal, presented herein as “low antibody” level.

The animal is preferably a mammal or an avian animal. Preferred mammals include a porcine, a bovine, an ovine, a caprine, a rodent, a swine and a human. Preferred avians include chickens, ducks, turkeys, quails, and other domesticated poultry.

The antibody can be produced in eggs by vaccinating a hen with CCK. Either purified CCK or synthesized CCK peptide can be used to vaccinate hens. Since CCK has a molecular weight of less than 1,500 Daltons, the CCK peptide should be conjugated with a carrier having a molecular weight of at least about 8,000 Daltons to elicit an immune response. Suitable carriers include a wide variety of conventional substances that include but are not limited to bovine gamma globulin and keyhole limpet hemocyanin. The CCK peptide conjugated to its carrier protein can be injected into the target animal with a common adjuvant. The CCK-carrier conjugate can be emulsified in an adjuvant such as Freund's complete adjuvant. If mammals are the target animals, then subsequent inoculations can be emulsified in incomplete adjuvant. Well known means in the art can be used for purifying the CCK peptide such as fractionization, chromatography, precipitation or extraction.

In response to exposure to the CCK antigen, the hens produce antibodies specific to CCK. The antibodies are then passively transferred into the egg yolk of eggs that are laid by the hens. Automated systems can separate and spray dry the yolks into a powder. The yolks can alternatively be lyophilized. Whole eggs may be used and it is therefore not necessary to separate the yolk from the albumen. Chickens are a preferred source of eggs but eggs from turkeys, geese, ducks and the like may also be used.

The source of the antibodies is not critical. While eggs are a preferred source of large quantities of antibodies, it is possible to collect the antibodies from whole blood, plasma or serum when chickens are processed for meat. In addition, whole blood, plasma or serum from inoculated livestock may be another source of antibodies as well as milk derived from an inoculated cow or goat. Additionally, another source of antibody production is through cell fusion using hybridoma techniques, genetically altered cell cultures or fermentation using recombinant technology.

In order to gauge the amount of antibody needed to achieve the desired effects, any well-established technique of measuring relative strength of different antibodies such as ELISA can be used so long as it is used consistently for different batches of antibodies.

Feeding is a preferred administration route since it is an efficient mechanism for delivering the treatment to many animals. Besides active feeding, other two modes of exposing animals to sufficiently high levels of anti-CCK antibodies to elicit similar responses of appetite suppression and weight gain reduction include passive transfer and active inoculation. With passive transfer, laying hens are injected with CCK, the hens produce antibodies specific to CCK and those antibodies are passively transferred into the egg yolk of eggs laid by the hens. The chick embryo absorbs the CCK antibody during embryonic development. Thereafter, the CCK antibodies circulating in the hatched chick's bloodstream as well as passed to the gastrointestinal tract. With active inoculation, CCK antibodies are directly injected into a target animal to elicit the desired response of suppressing appetite and reducing weight gain. The skilled artisan will appreciate that it is less preferred to require injection of large numbers of animals.

CCK has the same effects of increased GI motility and satiety inhibition in mammals and avians (Pekas and Trout, 1990). Mammalian species passively transfer antibodies to their progeny and respond to CCK autoimmunization as avians do. The dam's antibodies are also identical to those passively transferred to the progeny in avians as well as mammals. Therefore, the effects of actively fed and passively transferred CCK antibodies on satiety and weight gain resulting from CCK observed in avians should also be seen in mammals and vice versa.

The invention would benefit humans who are overweight or who have weight control problems. Additionally, individuals with overeating disorders would benefit from this invention because their food intake could be controlled.

The example of low and high dose of CCK antibody having the opposite effect on appetite and weight gain suggests that similar opposite effects may exist with antibodies specific to other gastrointestinal peptides or hormones that affect an animal's feeding behavior and digestion. For example, gastrin is involved in signaling acid secretion into the gut and has a trophic action on gastric mucosa leading to hyperplasia. A low dose antibody to gastrin could be used to decrease acid secretion in animals with gastric ulcers or in cases where there is gastric ELC cell carcinoid tumors due to prolonged hypergastrinemia while a high dose of the same antibody may elicit the opposite responses. Gut somatostatin inhibits food intake in fed animals as well as many other gut activities. A low dose of antibody to somatostatin could prevent its inhibitory activities while a high dose of the same antibody may enhance the inhibitory activity. Bombesin stimulates a release of CCK. One could hypothesize that inhibiting bombesin using a low dose of antibody specific to bombesin may result in responses similar to low dose of antibodies specific to CCK while a high dose of bombesin antibody can have a similar effect of a high dose of CCK antibody. Neuropeptide Y has been reported to be a stimulus in feeding. It may be possible to inhibit its activity and regulate obesity in animals prone to develop such problems with a low dose neuropeptide Y antibody while enhance weight gain with a high dose of the same antibody.

### EXAMPLE 1

### Dose Response of Anti-CCK Antibody on Food Intake and Weight Gain

Methods

Choleocystokinin (CCK-8) (Fragment 26-33 amide with sulfated tyrosine) was conjugated to bovine gamma globulin using glutaraldehyde and was emulsified with Freund's complete adjuvant (1:1) and injected (100 μg CCK) into laying hens. A second injection of the CCK-8 conjugate in Freund's incomplete adjuvant was injected 7 days after primary injection. Another group of control hens which did not receive the CCK injection was also used. Eggs from control and CCK immunized hens were collected after at least 21 days following the primary inoculation. Yolks were collected from the eggs (albumen was discarded) and control or anti-CCK yolks were separately pooled, frozen, then freeze dried.

The ELISA assay for anti-CCK antibodies in egg yolk is performed according to the following protocol: first, microtiter plates are coated with CCK-bovine gamma globulin conjugate in excess of anti-CCK to be tested by adding 100 μl of CCK solution in a concentration of 10 μg/ml to each well and storing the plates at 4° C. overnight or at room temperature for 5 hours. Then, the coated wells are washed three times with 200 μl 0.5% phosphate buffered saline (PBS)-Tween 20. Next, each coated well is blocked with bovine serum albumin (BSA) by adding 150 μl of 1% BSA in PBS and incubating at room temperature for 30 minutes. Extracted egg yolk antibody is serially diluted with water and then 100 μl of the serial diluted antibodies are added into each well (the egg yolk antibody is extracted by making a 10% solution of powered egg yolk in acidified water (pH=5), vigorously shaking the solution for two minutes, centrifuging the solution for 10 minutes and taking the aqueous part). The plates are incubated at room temperature for 30 minutes. Each well is then washed twice with 200 μl 0.5% PBS-Tween 20. One hundred μl of rabbit anti-chicken IgG labeled with alkaline phosphatase (1:2,000 dilution with the block solution or a dilution to assure that it is in excess of the anti-CCK to be tested) is added to each well and the plates are incubated at room temperature for 30 minutes. Each well is again washed twice with 200 μl 0.5% PBS-Tween 20. Then, 150 μl of substrate solution (97 ml diethanolamine, 100 mg MgCl₂, 0.2 g NaN₃, 800 ml water adjusted to pH 9.8 with 10 M Hcl, then adjusted to final volumne of 1 l with distilled water) is added in excess of bound enzymatic capacity into each well and the plates are incubated at room temperature for 15 minutes. The reaction is stopped by adding 50 μl of 6N NaOH to each well. Lastly, the absorbance is determined at 405 nm.

The amount of antibody in the dilution which gives an OD reading equal to two times the negative control well reading is defined as 1 titer unit.

The control and CCK antibody dried yolks were mixed with Basal Diets (University of Wisconsin Feed Mill) to reach the desired CCK antibody dose. Each group of 25 Broiler birds were fed with a diet containing a certain dose of CCK antibody. Animal weight gain and feed consumption were measured on day 0, 7, 14, and 21. Day 0 was the first day the birds were fed with yolk-containing diet.

Results

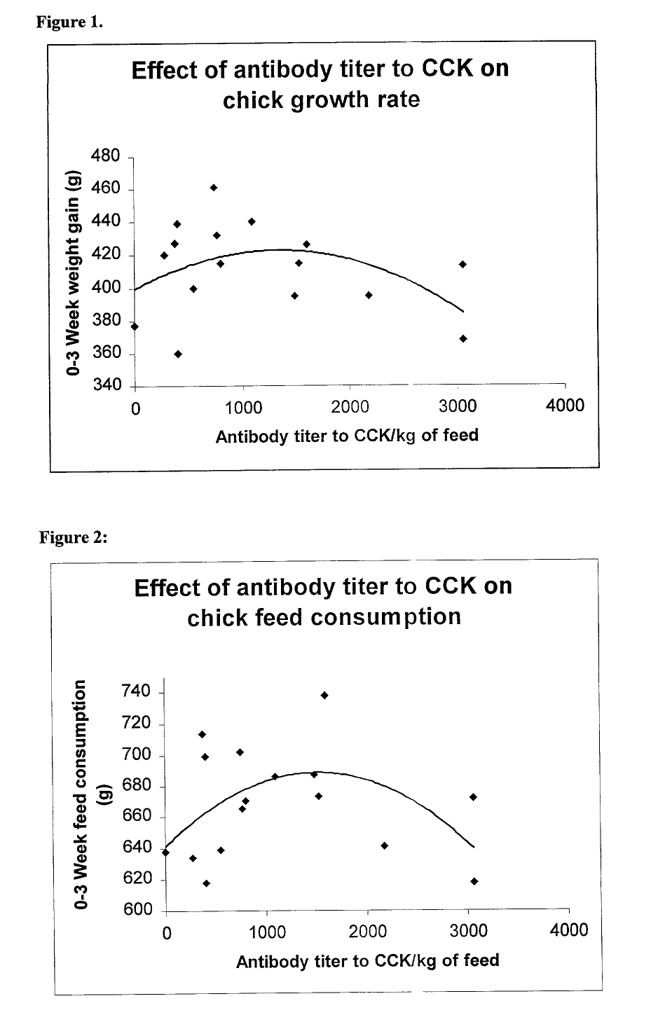

As shown in FIG. 1, low dose anti-CCK antibody in diet can increase body weight gain in Broiler birds. The effect of anti-CCK antibody to increase weight gain increases with higher antibody dosage until it peaks at a titer of about 1,300 units/kg of feed. After the peak titer, higher antibody dosage gradually loses the positive effect on body weight gain. In fact, any antibody dosage higher than a titer of about 2,700 units/kg of feed reduces body weight gain in comparison to that of the control Broiler birds.

FIG. 2 shows a similar effect on feed intake. Low dose antibody increases feed intake. Such a positive effect on feed intake peaks around an antibody titer of 1,500 units/kg of feed, above which the effect is gradually lost. Any antibody dosage higher than 3,000 units/kg of feed suppresses feed intake to below the control level.

### EXAMPLE 2

### Suppressing Appetite and Reducing Weight Gain in Pigs by Feeding Diets Containing Relatively High Doses of Anti-CCK Antibodies

Methods

Anti-CCK antibodies were generated in eggs, titers were determined and diet/egg yolk mixtures containing the desired antibody dose were made as described in example 1.

Four groups of eight pigs were fed, for three weeks, with a control diet, and a diet having an anti-CCK antibody titer per kg of feed of 5,936, 17,808 and 59,360, respectively. After three weeks, the last three groups of pigs were switched to diet having a titer of anti-CCK antibodies at 742, 2,256 and 7,420, respectively.

Results

Although these high dose CCK antibody diets increased weight gain in pigs initially between 1-2 week, Table 1 shows that diets having high anti-CCK antibody titer reduced weight gain in pigs between 2-3 week, which resulted a net reduced weight gain after 3 weeks of feeding. The same trend is observed for feed intake. As was noted in Example 1, it takes a higher titer of anti-CCK antibody to reduce feed intake than to suppress appetite. This is consistent with the result here that a titer of 5,963 is enough to reduce body weight gain but not to reduce feed intake between 2-3 week.

TABLE 1
Effects of feeding high dose levels of egg yolk
antibody to cholecystokinin to growing swine

Treatments

| | | | |
|---|---|---|---|
| During high | During low | High Antibody Period* | Low Antibody Period** |

| antibody period | antibody period | 2-3 Wk | 2-3 Wk feed | 3-4 Wk | 3-4 Wk feed |
|---|---|---|---|---|---|
| Antibody titer to CCK/kg feed | | gain (kg) | consumed (kg) | gain (kg) | consumed (kg) |
| 0 | 0 | 6.36 | 12.52 | 16.36 | 11.10 |
| 5936 | 742 | 5.91 | 12.52 | 17.27 | 18.21 |
| 17808 | 2256 | 5.45 | 11.61 | 16.36 | 20.22 |
| 59360 | 7420 | 4.54 | 11.51 | 15.90 | 22.39 |

*Period in which high doses of antibody were fed
**Period in which low doses of antibody were fed

The negative effect of high dose CCK antibody on weight gain and feed intake is reversible. As shown in tables and figures, once the pigs were switched from high dose antibody diets to relatively low dose antibody diets, they consumed more feed and gained more body weight (except the highest dose group) than control pigs.

We claim:

1. A method of suppressing appetite in an animal, comprising the step of administering to the animal a sufficient amount of an antibody to a gut peptide for a sufficiently long time to suppress appetite of the animal.

2. The method in claim 1 wherein the gut peptide is choleocystokinin.

3. The method in claim 1 wherein the gut peptide is bombesin.

4. The method in claim 1 wherein the gut peptide is somatostatin.

5. The method in claim 1 wherein the animal is an avian.

6. The method in claim 5 wherein the avian is a chicken.

7. The method in claim 1 wherein the animal is a mammal.

8. The method in claim 7 wherein the mammal is selected from the group consisting of a porcine, a bovine, an ovine, a caprine, a rodent, a swine and a human.

9. The method in claim 1 wherein the antibody is administered by feeding.

10. A method of suppressing appetite and reducing weight gain in animals, comprising the step of:
(1) immunizing a producer animal with a gut peptide so that the producer animal produces an antibody to the gut peptide;
(2) isolating a substance containing the gut peptide antibody from the producer animal; and
(3) feeding a sufficiently high dose of the substance containing the gut peptide antibody to an animal for a sufficiently long period of time.

11. The method in claim 10 wherein the gut peptide is choleocystokinin.

12. The method in claim 10 wherein the gut peptide is bombesin.

13. The method in claim 10 wherein the gut peptide is somatostatin.

14. The method in claim 10 wherein the animal is an avian.

15. The method in claim 14 wherein the avian is a chicken.

16. The method in claim 10 wherein the animal is a mammal.

17. The method in claim 16 wherein the mammal is selected from the group consisting of a porcine, a bovine, an ovine, a caprine, a rodent, a swine and a human.
